(19) United States
(12) Patent Application Publication
(10) Pub. No.: US 2024/0019454 A1
(43) Pub. Date: Jan. 18, 2024

(54) INTEGRATED AUTOMATED ANALYZER AND METHODS OF ANALYZING WHOLE BLOOD AND PLASMA FROM A SINGLE SAMPLE TUBE

(71) Applicant: BECKMAN COULTER, INC., Brea, CA (US)

(72) Inventors: Richard L. Situ, Placentia, CA (US); Lewis M. Mindeman, Anaheim, CA (US); Michael J. Calabrese, Brea, CA (US); Colin L. Granger, Chino Hills, CA (US); Kyle Suico, Brea, CA (US)

(21) Appl. No.: 18/254,636

(22) PCT Filed: Dec. 21, 2021
(86) PCT No.: PCT/US2021/064563

§ 371 Date: May 26, 2023

Related U.S. Application Data
(60) Provisional application No. 63/132,171, filed on Dec. 30, 2020.

(51) Int. Cl. G01N 35/04; G01N 33/49

(52) U.S. Cl. CPC G01N 35/04; G01N 33/491; G01N 2035/00495

(57) ABSTRACT

The presently claimed and described technology provides integrated automated instruments (100, 100A, 100B, 100C) and methods (800) for automatically processing a whole blood sample (50) in a single sample tube (80). These include: mixing the sample in the tube; analyzing a portion (50P) of the mixed sample (50M); centrifuging the sample in the tube thereby separating plasma (60); and analyzing a portion (60P) of the plasma. A pretreatment module (200) may mix, centrifuge, transport, and store the sample. The integrated instrument may include multiple detector components and does not require external sample handling.

### RELATED APPLICATIONS

This application claims priority to U.S. Provisional Patent Application Ser. No. 63/132,171 filed on Dec. 30, 2020, the entire disclosure of which is hereby incorporated by reference.

### FIELD

Various aspects of the presently disclosed and claimed technology relates to automated analyzers and methods for measuring analytes in liquid biological samples.

### BACKGROUND

Automated analyzers are commonly used in clinical chemistry, immunoassay, hematology, and other biological sampling and analyzing applications. Automated analytical equipment, such as automated analytical chemistry instruments, automated analytical immunoassay instruments, automated analytical hematology instruments, etc., can efficiently perform clinical analysis on a large number of samples, with multiple tests being run concurrently or within short time intervals. Efficiencies result, in part, because of the use of automated sample identification and tracking. This equipment can automatically prepare appropriate volume samples and can automatically set the test conditions needed to perform the scheduled tests. Test conditions can be independently established and tracked for different testing protocols simultaneously in process within a single analyzer, facilitating the simultaneous execution of a number of different tests based on different processes. Automated analytical instruments are particularly well-suited for high-volume and mid-volume testing environments, such as those existing in many hospitals and in centralized testing laboratories, because the automatic sample handling allows for more efficient sample identification and sample tracking. Automatic handling and tracking of samples significantly reduce the potential for human error or accidents that can lead to either erroneous test results or undesirable contamination.

However, the various types of tests may require differing starting sample matrixes such as whole blood, plasma and/or serum, and this leads to multiple samples being collected from the same patient. For example, the collection of whole blood samples and plasma samples are done in separate sample collection tubes because of compatibility issues between the collection tube used and the desired tests to be performed. These compatibility issues may include various anticoagulant compounds used in sample tubes to prevent the blood from coagulating and the interference of these compounds with the test to be performed. Thus, when gathering samples for analysis with automated analyzers, multiple sample tubes may be needed, depending on the tests desired. Even for a related set of tests for a given clinical condition, multiple sample tubes may be needed if the sample tubes required for the tests are incompatible with each other. For example, a related set of tests for a diabetes panel may include an HbA1c/T-Hb ratio, C-Peptide concentration, insulin concentration, and glucose concentration. As the HbA1c assay requires a whole blood sample, and others require plasma or serum samples, multiple sample tubes with multiple characteristics are used to collect multiple samples from the same patient for this diabetes panel.

Collecting patient samples in multiple tubes requires an increased total volume of the sample material from the patient. In certain environments, such as pediatric care, sample volume may be limited in availability.

In high-volume testing applications, a laboratory may be configured with a plurality of specific instrument types connected by an external conveyor/sample handling system. Multiple samples for a given patient test panel may be routed between the various instrument types to analyze the multiple samples. For mid-volume applications, the larger footprint, additional cost, and transit time of such an external conveyor system are disadvantages.

Further limitations and disadvantages of conventional and traditional approaches will become apparent to one of skill in the art, through comparison of such systems with some aspects of the present disclosure, as set forth in the remainder of the present application with reference to the drawings.

There is a need for a multi-component clinical analysis system that can analyze a single patient sample contained in a single sample tube with multiple tests and/or multiple test-types. There is further a need for a multi-component clinical analysis system that is integrated within a single compact housing that can perform multiple analysis types without an external conveyor system.

### BRIEF SUMMARY

The Inventors have recognized the need for a multi-component clinical analysis system that employs a single sample tube for holding a single patient sample that can be analyzed with multiple tests and/or multiple test-types. The Inventors have further recognized the need for an automated integrated multi-component clinical analysis system that does not require an external conveyor, robot, or other sample handling system.

According to certain aspects of the present disclosure, a method of automatically processing a whole blood sample with an integrated automated analyzer includes presenting the whole blood sample in a single sample tube with or without a cap to a pretreatment module. The integrated automated analyzer includes a detector arrangement with at least one detector and the pretreatment module. The pretreatment module includes a whole blood mixer, a centrifuge, and a transporter arrangement. The method further includes: delivering the single sample tube to the whole blood mixer with the transporter arrangement, mixing the whole blood sample in the single sample tube with the whole blood mixer, delivering a portion of the mixed whole blood sample from the single sample tube to the at least one detector with the transporter arrangement, analyzing the portion of the mixed whole blood sample with the at least one detector, delivering the single sample tube to the centrifuge with the transporter arrangement, centrifuging the whole blood sample in the single sample tube with the centrifuge and thereby separating plasma from the whole blood sample in the single sample tube, delivering a portion of the plasma from the single sample tube to the detector arrangement, and analyzing the portion of the plasma with the detector arrangement.

In a first aspect, the disclosure provides a method (800) of automatically processing a whole blood sample (50) with an integrated automated analyzer (100, 100A, 100B, 100C), the whole blood sample presented to a pretreatment module (200) of the integrated automated analyzer in a single sample tube (80) with or without a cap (90), the method comprising:
suppling the integrated automated analyzer, the integrated automated analyzer comprising:
   a detector arrangement (600) including at least one detector (420, 440, 520); and
   the pretreatment module, the pretreatment module comprising:
      a whole blood mixer (230);
      a centrifuge (260); and
      a transporter arrangement (700, 700A);
presenting the whole blood sample to the pretreatment module in the single sample tube;
delivering the single sample tube to the whole blood mixer with the transporter arrangement;
mixing the whole blood sample in the single sample tube with the whole blood mixer;
delivering a portion (50P) of the mixed whole blood sample (50, 50M) from the single sample tube to the at least one detector with the transporter arrangement;
analyzing the portion of the mixed whole blood sample with the at least one detector;
delivering the single sample tube to the centrifuge with the transporter arrangement;
centrifuging the whole blood sample in the single sample tube with the centrifuge and thereby separating plasma (60) from the whole blood sample in the single sample tube;
delivering a portion (60P) of the plasma from the single sample tube to the detector arrangement; and
analyzing the portion of the plasma with the detector arrangement.

In some aspects of the method, the at least one detector of the detector arrangement includes a photometer (420) for performing absorption photometry and wherein the portion of the mixed whole blood sample is analyzed with the photometer.

In some aspects of the method, the method further comprises: pipetting the portion of the mixed whole blood sample from the single sample tube to a first vessel (40, 40₁) of the integrated automated analyzer with the transporter arrangement; pipetting a hemolyzing reagent (20₁) into the first vessel with the transporter arrangement thereby producing hemolyzed whole blood; pipetting a first portion of the hemolyzed whole blood from the first vessel to a second vessel (40, 40₂) of the integrated automated analyzer with the transporter arrangement; pipetting a second portion of the hemolyzed whole blood from the first vessel to a third vessel (40, 40₃) of the integrated automated analyzer with the transporter arrangement; pipetting a Total Hemoglobin (T-Hb) reagent (20₂) into the second vessel with the transporter arrangement; pipetting an HbA1c reagent (20₃) into the third vessel with the transporter arrangement; determining a Total Hemoglobin (T-Hb) concentration corresponding to the whole blood sample by applying a colorimetric method to the second vessel with the photometer; determining an HbA1c concentration corresponding to the whole blood sample by applying a turbidimetric immunoinhibition method to the third vessel with the photometer; and reporting an HbA1c/T-Hb ratio corresponding to the whole blood sample.

In some aspects of the method, the at least one detector of the detector arrangement includes a photometer (420) for performing absorption photometry and wherein the portion of the plasma is analyzed with the photometer.

In some aspects of the method, the method further comprises pipetting the portion of the plasma from the single sample tube to a vessel (40) of the integrated automated analyzer with the transporter arrangement; pipetting a glucose reagent (20) into the vessel with the transporter arrangement; determining a glucose concentration corresponding to the whole blood sample by applying a colorimetric method to the vessel with the photometer; and reporting a quantitative determination of glucose levels corresponding to the whole blood sample.

In some aspects of the method, the single sample tube comprises an additive selected from the group consisting of sodium heparin, lithium heparin, and ethylenediaminetetraacetic acid (EDTA).

In some aspects of the method, the at least one detector of the detector arrangement includes a luminometer (520) and wherein the portion of the plasma is analyzed with the luminometer.

In some aspects of the method, the method further comprises: pipetting the portion of the plasma from the single sample tube to a vessel (30) of the integrated automated analyzer with the transporter arrangement; pipetting a C-Peptide reagent (20) into the vessel with the transporter arrangement; determining a C-Peptide concentration corresponding to the whole blood sample by applying a reagent capture luminescence method about the vessel with the luminometer; and reporting a quantitative assessment of an ability of pancreatic beta cells to secrete insulin corresponding to the whole blood sample.

In some aspects of the method, the single sample tube comprises a lithium heparin or EDTA additive.

In some aspects of the method, the additive is lithium heparin and the at least one detector of the detector arrangement includes an ion selective electrode (ISE) (440) for performing electrolyte analysis.

In some aspects of the method, the method further comprises: pipetting the portion of the plasma from the single sample tube to a vessel (30) of the integrated automated analyzer with the transporter arrangement; pipetting an Insulin reagent (20) into the vessel with the transporter arrangement; determining an insulin concentration corresponding to the whole blood sample by applying a reagent capture luminescence method about the vessel with the luminometer; and reporting a quantitative determination of insulin levels corresponding to the whole blood sample.

In some aspects of the method, the single sample tube comprises an EDTA additive.

In some aspects of the method, results from analyzing the portion of the mixed whole blood sample with the at least one detector are automatically used in determining details of analyzing the portion of the plasma with the detector arrangement.

In some aspects of the method, the pretreatment module further comprises a capper (290) configured to apply the cap on the single sample tube, the method further comprising: applying the cap on the single sample tube prior to mixing the whole blood sample in the single sample tube with the whole blood mixer.

In some aspects of the method, wherein the pretreatment module further comprises a capper (290) configured to apply the cap on the single sample tube, the method further comprising: applying the cap on the single sample tube prior to centrifuging the whole blood sample in the single sample tube with the centrifuge.

In some aspects of the method, the pretreatment module further comprises a decapper (320) configured to remove the cap from the single sample tube, the method further comprising: removing the cap from the single sample tube prior to delivering the portion of the mixed whole blood sample from the single sample tube to the at least one detector with the transporter arrangement.

In some aspects of the method, the pretreatment module further comprises a decapper (320) configured to remove the cap from the single sample tube, the method further comprising: removing the cap from the single sample tube prior to delivering the portion of the plasma from the single sample tube to the detector arrangement.

In some aspects of the method, the pretreatment module further comprises a reader (390) configured to identify the single sample tube, the method further comprising: reading an identity of the single sample tube after presenting the whole blood sample to the pretreatment module in the single sample tube.

In some aspects of the method, the integrated automated analyzer further comprises at least one rack (70) and wherein the whole blood sample is presented to the pretreatment module of the integrated automated analyzer with the single sample tube in the rack.

In some aspects of the method, the pretreatment module further comprises at least one storage location (350) configured to store the single sample tube, the method further comprising: storing the single sample tube at the storage location after presenting the whole blood sample to the pretreatment module in the single sample tube.

In some aspects of the method, the single sample tube is stored capped.

In some aspects of the method, the pretreatment module further comprises at least one storage location (350) configured to store the single sample tube, the method further comprising: storing the single sample tube at the storage location after delivering the portion of the mixed whole blood sample from the single sample tube to the at least one detector with the transporter arrangement.

In some aspects of the method, the pretreatment module further comprises at least one storage location (350) configured to store the single sample tube, the method further comprising: storing the single sample tube at the storage location after delivering the portion of the plasma from the single sample tube to the detector arrangement.

In some aspects of the method, the integrated automated analyzer further comprises at least one rack (70), the method further comprising: storing the single sample tube in the rack at the storage location.

In some aspects of the method, the at least one detector of the detector arrangement includes an ion selective electrode (ISE) (440) for performing electrolyte analysis.

These and other advantages, aspects, and novel features of the present disclosure, as well as details of illustrated embodiments thereof, will be more fully understood from the following description and drawings.

### BRIEF DESCRIPTION OF THE FIGURES

Embodiments of the present disclosure will now be described, by way of example only, with reference to the attached Figures, wherein:

FIG. 1 is a schematic configuration diagram illustrating an integrated automated analyzer, in accordance with the principles of the present disclosure;

FIG. 2 is a schematic configuration diagram illustrating an example detector arrangement of the integrated automated analyzer of FIG. 1, in accordance with the principles of the present disclosure;

FIG. 3 is a schematic configuration diagram illustrating an example transporter arrangement of the integrated automated analyzer of FIG. 1, in accordance with the principles of the present disclosure;

FIG. 4 is a schematic configuration diagram illustrating another integrated automated analyzer, in accordance with the principles of the present disclosure;

FIG. 5 is a schematic configuration diagram illustrating still another integrated automated analyzer, in accordance with the principles of the present disclosure;

FIG. 6 is a schematic configuration diagram illustrating a pretreatment module of the integrated automated analyzers of FIGS. 1, 4, and 5, in accordance with the principles of the present disclosure;

FIG. 7 is a single sample tube suitable for use with the integrated automated analyzers of FIGS. 1, 4, and 5, in accordance with the principles of the present disclosure, the single sample tube illustrated with an unmixed whole blood sample therein;

FIG. 8 is the single sample tube of FIG. 7 illustrated with a mixed whole blood sample therein;

FIG. 9 is the single sample tube of FIG. 7 illustrated with a centrifuged whole blood sample therein;

FIG. 10 is a perspective view of a rack suitable for holding the single sample tube of FIG. 7 and suitable for use in presenting the single sample tube to the integrated automated analyzers of FIGS. 1, 4, and 5;

FIG. 11 is a perspective view of a vessel suitable for use within the integrated automated analyzers of FIGS. 1 and 4;

FIG. 12 is a cross-sectional elevation view of a vessel suitable for use within the integrated automated analyzers of FIGS. 1 and 5;

FIG. 13 is a schematic configuration diagram illustrating certain interactions of the example transporter arrangement of FIG. 3, in accordance with the principles of the present disclosure; and

FIG. 14 is a flow chart illustrating a method of operating the integrated automated analyzers of FIGS. 1 and 4.

### DETAILED DESCRIPTION

Various embodiments will be described in detail with reference to the drawings, wherein like reference numerals represent like parts and assemblies throughout the several views. It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a”, “an”, and “the” include plural reference unless the context clearly indicates otherwise.

The term “about” is used in connection with a numerical value throughout the specification and the claims denote an interval of accuracy, familiar and acceptable to a person skilled in the art. In general, such an interval of accuracy is +/−10%.

Although certain assays referenced herein may correspond to certain commercially available assays, the references are intended to be generic and not limited to any specific commercial assay.

Unless defined otherwise, all technical and scientific terms used herein have the same meaning as commonly understood to one of ordinary skill in the art to which this disclosure belongs.

According to the principles of the present disclosure, a pretreatment module (i.e., a pre-analytic module) is adapted to be integrated with one or more clinical automated analyzers. The pretreatment module serves to further automate automated clinical sample analysis by preparing a patient sample for one or more analyses by the one or more clinical automated analyzers. Example functions performed by the pretreatment module include: whole blood mixing, centrifuging, capping, decapping, storage, sample identification, and transportation of the patient sample—both within a container (e.g., via a pick-and-place arrangement) and as a fluid outside of a container (e.g., via a pipettor arrangement).

In certain aspects, a pretreatment module (i.e., a pre-analytic module) serves to connect at least two clinical automated analyzers and thereby facilitate and coordinate patient sample testing between the clinical automated analyzers.

According to the principles of the present disclosure, a single sample tube that contains a single patient sample may be used in several assay types and/or in several assays, thereby reducing the required overall volume of the patient sample, reducing the required time that the patient must endure a blood draw, reducing the required number of sample tubes, reducing the required storage area when storing the patient sample, and/or other benefits.

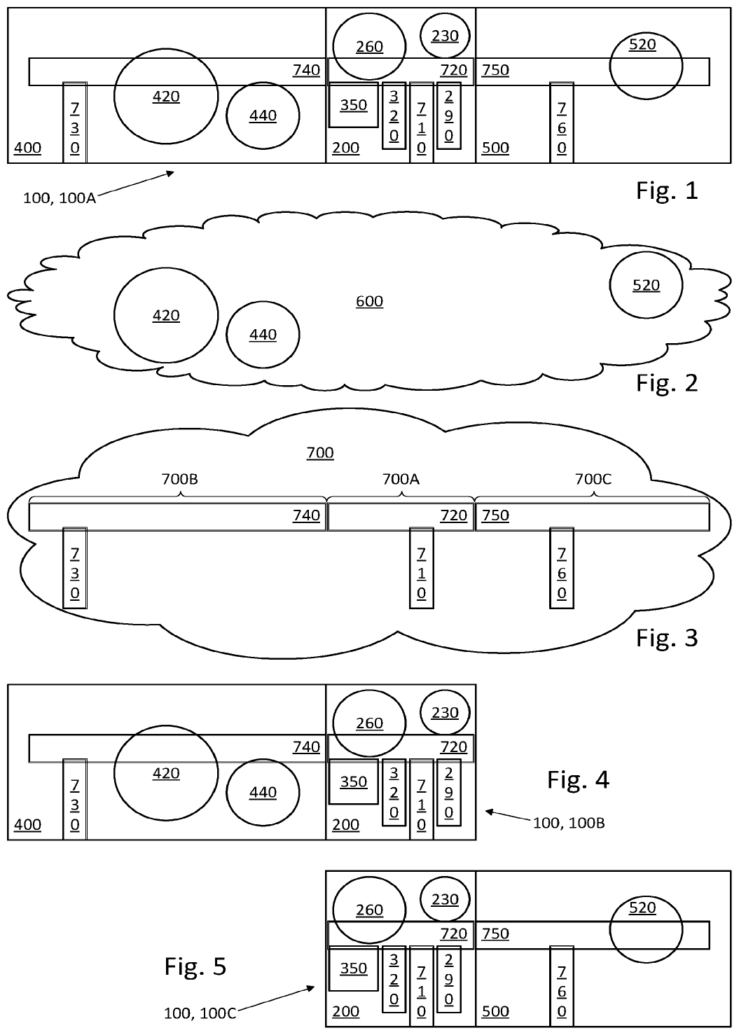

Turning now to FIG. 1, an integrated automated analyzer 100, 100A is illustrated according to the principles of the present disclosure. As depicted, the integrated automated analyzer 100A includes a pretreatment module 200, a first analyzing module 400, and a second analyzing module 500. The first analyzing module 400 may include clinical chemistry analyzing capabilities. As depicted, the first analyzing module 400 includes a first detector 420 and a second detector 440. The first detector 420 may include a photometer, and the second detector 440 may include ion selective electrodes (ISE). The second analyzing module 500 may include immunoassay analyzing capabilities. As depicted, the second analyzing module 500 includes a detector 520. The detector 520 may include a luminometer. In other embodiments, other types of analyzing modules may be added or may be substituted for the analyzing modules 400 and/or 500. For example, a module with hematology analyzing capabilities may be added or substituted.

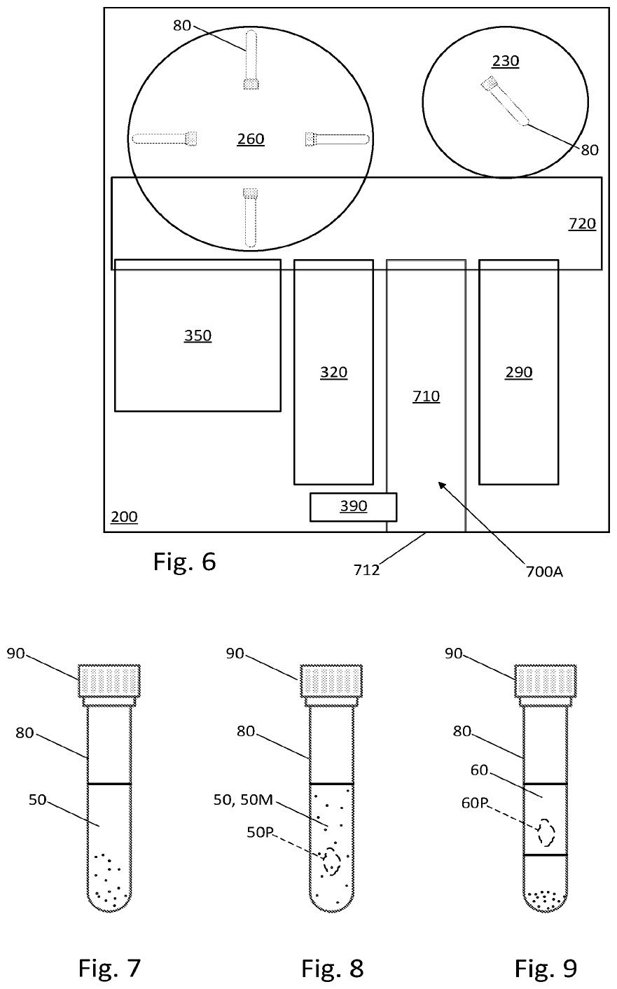

Turning now to FIG. 6, the pretreatment module 200 will now be described in further detail. As depicted, the pretreatment module 200 includes a whole blood mixer 230, a centrifuge 260, a capper 290, a decapper 320, a storage location 350, a reader 390, and a transporter arrangement 700A. As depicted, the transporter arrangement 700A includes an infeed-outfeed 710 and a transporter 720.

Turning now to FIGS. 7-9, a sample tube 80 is illustrated. In the depicted embodiment, the sample tube 80 includes a cap 90. In other embodiments, the sample tube 80 may not necessarily include a cap. In some embodiments, the sample tube contains an additive. The additive may be an anticoagulant. Non-limiting examples of anticoagulants include ethylenediaminetetraacetic acid (EDTA), sodium citrate, CTAD (citrate, theophylline, adenosine, and dipyridamole), lithium heparin, sodium heparin, sodium fluoride, acid citrate dextrose, and sodium polyanethol sulfonate.

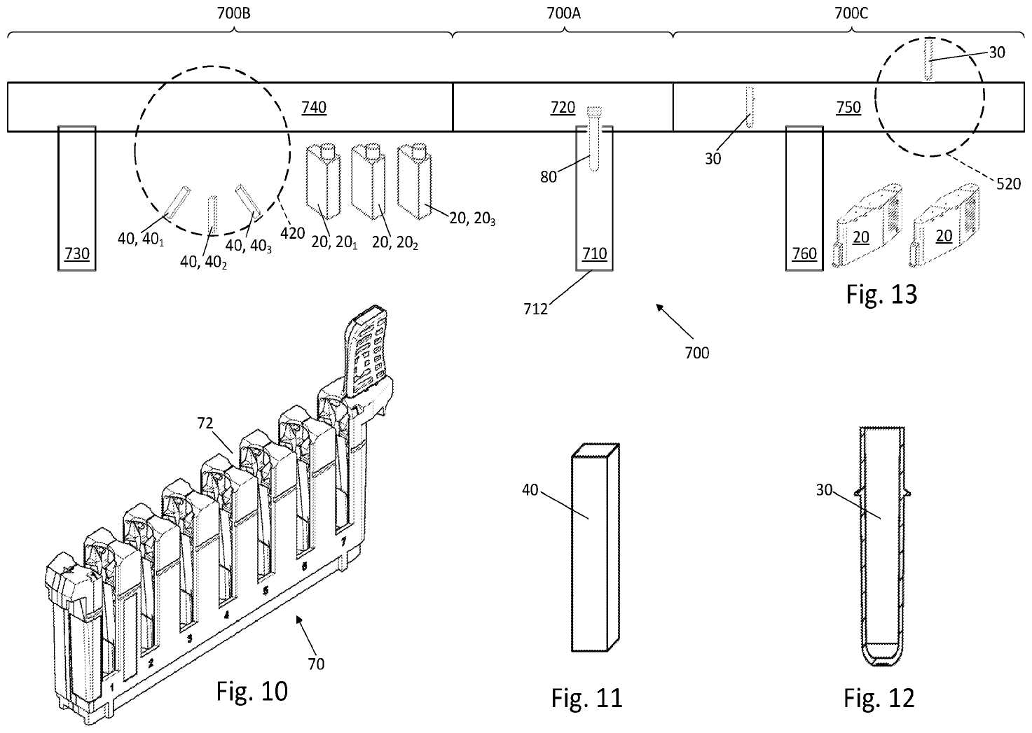

Turning now to FIG. 10, a rack 70 is illustrated. The rack 70 is configured to hold a plurality of the sample tubes 80. In particular, a plurality of tube holders 72 of the rack 70 are configured to each hold one of the sample tubes 80.

Turning again to FIG. 6, the pretreatment module 200 will now be described in further detail. The pretreatment module 200 may receive one or more of the sample tubes 80. In certain embodiments, the sample tube 80 is presented directly to an inlet-outlet 712 of the infeed-outfeed 710. In other embodiments, the sample tube 80 is positioned in a rack, such as the rack 70, and the rack 70 is presented to the infeed-outfeed 710 at the inlet-outlet 712. In still other embodiments, the sample tube 80 may either be individually presented to the inlet-outlet 710 or may be presented to the inlet-outlet 712 within the rack 70.

Upon receiving the sample tube 80, the transporter arrangement 700A may distribute the sample tube 80 according to a testing sequence appropriate for the desired test(s) of a specimen within the sample tube 80. The reader 390 may read an identifying mark on the sample tube 80 and thereby identify the specimen within the sample tube 80 and further identify the testing sequence for the sample tube 80. The reader 390 may further detect whether the sample tube 80 is fitted with a cap, such as the cap 90.

An example testing sequence may include mixing of a whole blood sample 50 by the whole blood mixer 230. Upon presentation of the sample tube 80 with the whole blood sample 50 therein, the reader 390 may identify the specimen 50 within the sample tube 80. According to the schedule of the integrated automated analyzer 100, the sample tube 80 may be delivered to the storage location 350 for processing at a later time, or the sample tube 80 may be transferred by the transporter arrangement 700A to the whole blood mixer 230. If the sample tube 80 is without a cap, the transporter arrangement 700A may transport the sample tube 80 to the capper 290, and the capper 290 may apply a cap 90 to the sample tube 80. Upon the sample tube 80 receiving the cap 90, the sample tube 80 may proceed to the whole blood mixer 230. Upon the sample tube 80 being received by the whole blood mixer 230, the whole blood mixer 230 mixes the whole blood sample 50 within the sample tube 80. Upon the whole blood sample 50 being mixed, the mixed whole blood sample 50M may be delivered to one of the modules 400, 500 for analysis.

An example testing sequence may include centrifuging of a whole blood sample 50 by the whole blood mixer 230. Upon presentation of the sample tube 80 with the whole blood sample 50 therein, the reader 390 may identify the specimen 50 within the sample tube 80. According to the schedule of the integrated automated analyzer 100, the sample tube 80 may be delivered to the storage location 350 for processing at a later time, or the sample tube 80 may be transferred by the transporter arrangement 700A to the centrifuge 260. If the sample tube 80 is without a cap, the transporter arrangement 700A may transport the sample tube 80 to the capper 290, and the capper 290 may apply a cap 90 to the sample tube 80. Upon the sample tube 80 receiving the cap 90, the sample tube 80 may proceed to the centrifuge 260. Upon the sample tube 80 being received by the centrifuge 260, the centrifuge 260 centrifuges the whole blood sample 50 within the sample tube 80. Upon the whole blood sample 50 being centrifuged, plasma 60 separated from the whole blood sample 50 by the centrifuge 260 may be delivered to one of the modules 400, 500 for analysis.

Turning now to FIGS. 3 and 13, a transporter arrangement 700 of the integrated automated analyzer 100 will be described in detail. The transporter arrangement 700 is configured to deliver patient samples, reagents, substrates, sample tubes, vessels, racks, and/or other liquids and containers throughout the integrated automated analyzer 100. Thus, various liquids and/or containers may be moved between the various components and storage locations of the integrated automated analyzer 100.

The transporter arrangement 700 may include a pick-and-place arrangement with one or more pick-and-place devices. The pick-and-place devices may be configured to pick-up, move, and place-down the various containers of the integrated automated analyzer 100. As samples, reagents, substrates, and/or other liquids may be present in the various containers, the pick-and-place arrangement may be used to transfer various liquids within and throughout the integrated automated analyzer 100 and between the various components and storage locations of the integrated automated analyzer 100.

The transporter arrangement 700 may include a pipettor arrangement with one or more pipettors. The pipettors may be configured to aspirate and/or dispense the various liquids used within the integrated automated analyzer 100. The pipettors may be configured to aspirate and/or dispense the various liquids among and between the various containers of the integrated automated analyzer 100.

As illustrated at FIGS. 3 and 13, the transporter arrangement 700 may be configured as a first transporter arrangement 700A, a second transporter arrangement 700B, and/or a third transporter arrangement 700C. The first transporter arrangement 700A may be configured to primarily service the pretreatment module 200. The second transporter arrangement 700B may be configured to primarily service the first module 400. And, the third transporter arrangement 700C may be configured to primarily service the second module 500. The transporter arrangements 700A, 700B, and/or 700C may be configured to interoperate and thereby pass liquids, containers, racks, etc., amongst each other. In certain embodiments, the pick-and-place devices may transcend one or more of the transporter arrangements 700A, 700B, 700C. In certain embodiments, the pipettors may transcend one or more of the transporter arrangements 700A, 700B, 700C. In certain embodiments, one or more individual pick-and-place devices may only operate within one of the transporter arrangements 700A, 700B, or 700C. In certain embodiments, one or more individual pipettors may only operate within one of the transporter arrangements 700A, 700B, or 700C.

As mentioned above, the transporter arrangement 700A includes the infeed-outfeed 710 and the transporter 720. Similarly, the transporter arrangement 700B may include an infeed-outfeed 730 and a transporter 740. Similarly, the transporter arrangement 700C may include an infeed-outfeed 760 and a transporter 750. The infeeds-outfeeds 710, 730, 760 may include pick-and-place devices, pipettors, conveyors, and/or other elements suitable for the transportation of containers or fluids. The transporters 720, 740, 750 may include pick-and-place devices, pipettors, conveyors, and/or other elements suitable for the transportation of containers or fluids. The infeed-outfeed 710 and the transporter 720 may operate generally within the pretreatment module 200. The infeed-outfeed 730 and the transporter 740 may operate generally within the first module 400. And, the infeed-outfeed 760 and the transporter 750 may operate generally within the second module 500.

Turning now to FIG. 13, the transporter 700B will be described in further detail. The transporter 700B may be configured to transfer reagents 20, 20₁, 20₂, 20₃ to one or more cuvettes 40, further illustrated at FIG. 11. The transporter 700, 700A, 700B may further be configured to transfer samples to one or more of the cuvettes 40. Substrates, diluents, detergents, and/or other fluids may further be transferred to the cuvettes 40 by the transporter 700, 700A, 700B. Upon the cuvettes 40 being loaded with the appropriate materials, the detectors 420 and/or 440 may analyze the sample within the cuvette 40.

Turning again to FIG. 13, the transporter 700C will be described in further detail. The transporter 700C may be configured to transfer reagents 20 to one or more cuvettes 30, further illustrated at FIG. 12. The transporter 700, 700A, 700C may further be configured to transfer samples to one or more of the cuvettes 30. Substrates, diluents, detergents, and/or other fluids may further be transferred to the cuvettes 40 by the transporter 700, 700A, 700C. Upon the cuvettes 30 being loaded with the appropriate materials, the detector 520 may analyze the sample within the cuvette 30.

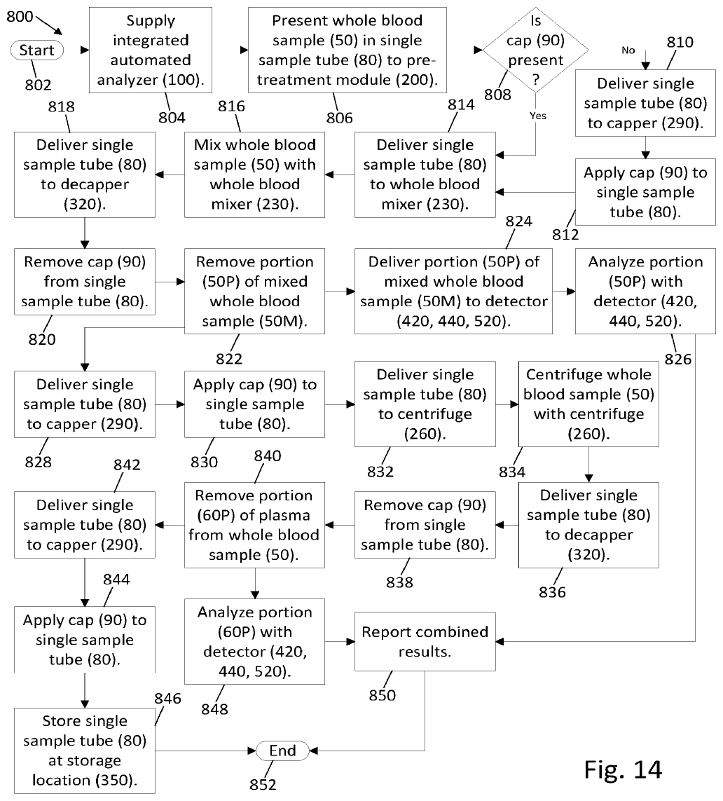

Turning now to FIG. 14, a method 800 of automatically processing a whole blood sample 50 with the integrated automated analyzer 100, 100A, 100B will be described according to the principles of the present disclosure. The method 800 employees a single sample tube 80. Multiple tests and multiple types of tests are thus done on a single sample 50 presented to the integrated automated analyzer 100, 100A, 100B in the single sample tube 80.

The method 800 begins at start 802. The integrated automated analyzer 100 is provided at step 804. The whole blood sample 50 is presented in the single sample tube 80 to the pretreatment module 200 at step 806. A test is done at step 808 to determine if the cap 90 is present on the single sample tube 80. If the cap 90 is present, then the single sample tube 80 is delivered to the whole blood mixer 230 at step 814. If the cap 90 is not present, then the single sample tube 80 is delivered to the capper 290 at step 810. From step 810, the cap 90 is applied to the single sample tube at step 812, and step 814 is performed thereafter.

Upon the single sample tube 80 being delivered to the whole blood mixer 230, the whole blood sample 50 is mixed with the whole blood mixer 230 at step 816. The single sample tube 80 is then delivered to the decapper 320 at step 818. At the decapper 320, the cap 90 is removed from the single sample tube 80 at step 820. Upon the cap 90 being removed from the single sample tube 80, a portion 50P of the mixed whole blood sample 50M is removed from the single sample tube 80 at step 822.

Upon the removal of the portion 50P from the single sample tube 80 at step 822, the method 800 may proceed in parallel to steps 824 and 828, in certain embodiments. In other embodiments, steps 824 and 826 are performed first, and step 828 is performed subsequently, depending on the results of step 826. At step 824, the portion 50P of the mixed whole blood sample 50M is delivered to the detector 420. The portion 50P is then analyzed with the detector 420 at step 826.

At step 828, the single sample tube 80 is delivered to the capper 290. Upon delivery to the capper 290, the cap 90 is applied to the single sample tube 80 at step 830. Upon the single sample tube 80 being capped, the single sample tube 80 is delivered to the centrifuge 260 at step 832. At step 834, the whole blood sample 50 is centrifuged with the centrifuge 260. Upon centrifuging the whole blood sample 50, the single sample tube 80 is delivered to the decapper 320 at step 836. Upon delivery to the decapper 320, the cap 90 is removed from the single sample tube 80 at step 838. Upon the cap 90 being removed, a portion 60P of plasma is removed from the whole blood sample 50 at step 840.

Upon the removal of the portion 60P of plasma from the single sample tube 80 at step 840, the method 800 may proceed in parallel to steps 842 and 848, in certain embodiments. In other embodiments, the steps 842 and 848 may be done in series with either being done first.

At step 842, the single sample tube 80 is delivered to the capper 290. Upon the single sample tube 80 being delivered to the capper 290, the cap 90 is applied to the single sample tube 80 at step 844. The capped single sample tube 80 may be stored at the storage location 350 at step 846.

Upon the portion 60P of plasma being removed from the single sample tube 80, the portion 60P may be analyzed with the detector 420, 440, 520 at step 848. Upon steps 848 and 826 being complete, combined results of the tests of steps 826 and 848 are reported at step 850. Upon steps 846 and 850 being complete, the end 852 of the method 800 is reached.

Turning now to FIG. 2, a detector arrangement 600 is illustrated according to the principles of the present disclosure. The detector arrangement 600 includes the first detector 420 and the second detector 440 of the first analyzing module 400 and also includes the detector 520 of the second analyzing module 500. As mentioned above, the first detector 420 may include the photometer, the second detector 440 may include the ion selective electrodes (ISE), and the detector 520 may include the luminometer. The detector arrangement 600 is included in the integrated automated analyzer 100, 100A. The integrated automated analyzer 100, 100A, 100B, 100C includes detectors, such as detectors 420, 440, and 520, within a single compact housing that can perform multiple analysis types without an external conveyor and/or transporting system.

Turning now to FIG. 4, the integrated automated analyzer 100, 100B is illustrated according to the principles of the present disclosure. The integrated automated analyzer 100B is similar to the integrated automated analyzer 100A, but with the analyzing module 500 removed.

Turning now to FIG. 5, the integrated automated analyzer 100, 100C is illustrated according to the principles of the present disclosure. The integrated automated analyzer 100C is similar to the integrated automated analyzer 100A, but with the analyzing module 400 removed.

Additional examples are provided below.

### EXAMPLES

### Example 1: Preparation of a Whole Blood Sample for Analysis Using an Anticoagulant Sample Tube

A whole blood sample was collected by drawing blood into a sample tube containing an anticoagulant. The collected whole blood samples were stable for up to 8 hours when stored at 20° C. to 25° C., 7 days when stored at 2° C. to 8° C., and up to 1 month when frozen at −20° C. to −70° C.

After collection, the whole blood sample was automatically processed using an integrated automated analyzer containing a photometer and ion selective electrode on a clinical chemistry analyzer and a luminometer on an immunoassay analyzer. After ensuring a cap is present on the sample tube, the anticoagulant containing sample tube was delivered to and mixed using a whole blood mixer. The anticoagulant-containing sample tube was then delivered to a decapper to remove the cap. Once the cap was removed, a portion of the mixed whole blood sample was removed from the anticoagulant-containing sample tube for further analysis.

### Example 2: Hemoglobin A1C and Glucose Diabetes Panel Testing Using a Whole Blood Sample

A diabetes panel combines at least two sample tests to provide a comprehensive assessment of blood sugar levels and can be used to diagnose diabetes or monitor diabetes treatment. The most simple diabetes panel consists of testing for hemoglobin A1C (HbA1c) and glucose. Advantages of an integrated testing approach allow for the ability to analyze both whole blood and plasma and ensure that the plasma is separated from blood cells as soon as possible, avoiding hemolysis. The whole blood sample was collected into a sample tube containing sodium heparin, lithium heparin, or EDTA and prepared for further analysis (Example 1).

HbA1c testing: The portion of the mixed whole blood sample and a Hemolyzing reagent were pipetted into a first vessel. A portion of this hemolyzed whole blood was then pipetted into a second and third vessel. Total Hemoglobin (T-Hb) reagent was then pipetted into the second vessel and HbA1c reagent into the third vessel. The photometer was then used to determine a Total Hemoglobin (T-Hb) concentration corresponding to the whole blood sample by applying a colorimetric method to the second vessel and a hemoglobin A1c (HbA1c) concentration corresponding to the whole blood sample by applying a turbidimetric immunoinhibition method to the third vessel. An HbA1c/T-Hb ratio corresponding to the whole blood sample was reported.

Glucose testing: After the portion of the mixed whole blood sample was removed for the HbAc1 testing, the sample tube was delivered to a centrifuge, and the plasma was separated from the whole blood by centrifuging the mixed whole blood sample. A portion of the plasma was pipetted from the centrifuged sample tube to a vessel and a glucose reagent was then added. The photometer was then used to determine a glucose concentration corresponding to the whole blood sample by applying a colorimetric method to the vessel. A quantitative determination of glucose levels was reported.

### Example 3: Hemoglobin A1C, Glucose, C-Peptide, and Ion Concentration Diabetes Panel Testing Using a Whole Blood Sample

The whole blood sample was collected into a sample tube containing lithium heparin and prepared for further analysis (Example 1). The HbA1c and glucose testing were the same as in Example 2.

C-Peptide: A portion of the plasma was pipetted from the centrifuged sample tube to a vessel and a C-Peptide reagent was pipetted into the vessel. The luminometer was then used to determine a C-Peptide concentration corresponding to the whole blood sample by applying a reagent capture luminescence method about the vessel. A quantitative assessment of an ability of pancreatic beta cells to secrete insulin corresponding to the whole blood sample was reported.

Ion Concentration: A portion of the plasma was pipetted from the centrifuged sample tube to a vessel. Using an ion selective electrode for Na⁺, K⁺, and Cl⁻, the concentrations of these ions were measured and a quantitative determination of Na⁺, K⁺, and Cl⁻ ion levels were reported.

### Example 4: Hemoglobin A1C, Glucose, C-Peptide, and Insulin Diabetes Panel Testing Using a Whole Blood Sample

The whole blood sample was collected into a sample tube containing EDTA and prepared for further analysis (Example 1). The HbA1c and glucose testing were the same as in Example 2, and the C-Peptide testing was the same as Example 3.

Insulin: A portion of the plasma was pipetted from the centrifuged sample tube to a vessel and an insulin reagent was pipetted into the vessel. The luminometer was then used to determine an insulin concentration corresponding to the whole blood sample by applying a reagent capture luminescence method about the vessel. A quantitative determination of insulin levels corresponding to the whole blood sample was reported.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred.

What is claimed is:

1. A method (800) of automatically processing a whole blood sample (50) with an integrated automated analyzer (100, 100A, 100B, 100C), the whole blood sample presented to a pretreatment module (200) of the integrated automated analyzer in a single sample tube (80) with or without a cap (90), the method comprising:
suppling the integrated automated analyzer, the integrated automated analyzer comprising:
a detector arrangement (600) including at least one detector (420, 440, 520); and
the pretreatment module, the pretreatment module comprising:
a whole blood mixer (230);
a centrifuge (260); and
a transporter arrangement (700, 700A);
presenting the whole blood sample to the pretreatment module in the single sample tube;
delivering the single sample tube to the whole blood mixer with the transporter arrangement;
mixing the whole blood sample in the single sample tube with the whole blood mixer;
delivering a portion (50P) of the mixed whole blood sample (50, 50M) from the single sample tube to the at least one detector with the transporter arrangement;
analyzing the portion of the mixed whole blood sample with the at least one detector;
delivering the single sample tube to the centrifuge with the transporter arrangement;
centrifuging the whole blood sample in the single sample tube with the centrifuge and thereby separating plasma (60) from the whole blood sample in the single sample tube;
delivering a portion (60P) of the plasma from the single sample tube to the detector arrangement; and
analyzing the portion of the plasma with the detector arrangement.

2. The method of claim 1, wherein the at least one detector of the detector arrangement includes a photometer (420) for performing absorption photometry and wherein the portion of the mixed whole blood sample is analyzed with the photometer.

3. The method of claim 2, further comprising:
pipetting the portion of the mixed whole blood sample from the single sample tube to a first vessel (40, 40₁) of the integrated automated analyzer with the transporter arrangement;
pipetting a hemolyzing reagent (20₁) into the first vessel with the transporter arrangement thereby producing hemolyzed whole blood;
pipetting a first portion of the hemolyzed whole blood from the first vessel to a second vessel (40, 40₂) of the integrated automated analyzer with the transporter arrangement;
pipetting a second portion of the hemolyzed whole blood from the first vessel to a third vessel (40, 40₃) of the integrated automated analyzer with the transporter arrangement;
pipetting a Total Hemoglobin (T-Hb) reagent (20₂) into the second vessel with the transporter arrangement;
pipetting an HbA1c reagent (20₃) into the third vessel with the transporter arrangement;
determining a Total Hemoglobin (T-Hb) concentration corresponding to the whole blood sample by applying a colorimetric method to the second vessel with the photometer;
determining an HbA1c concentration corresponding to the whole blood sample by applying a turbidimetric immunoinhibition method to the third vessel with the photometer; and
reporting an HbA1c/T-Hb ratio corresponding to the whole blood sample.

4. The method of claim 1, wherein the at least one detector of the detector arrangement includes a photometer (420) for performing absorption photometry and wherein the portion of the plasma is analyzed with the photometer.

5. The method of claim 4, further comprising:
pipetting the portion of the plasma from the single sample tube to a vessel (40) of the integrated automated analyzer with the transporter arrangement;
pipetting a Glucose reagent (20) into the vessel with the transporter arrangement;
determining a glucose concentration corresponding to the whole blood sample by applying a colorimetric method to the vessel with the photometer; and
reporting a quantitative determination of glucose levels corresponding to the whole blood sample.

6. The method of any of claims 1-5, wherein the single sample tube comprises an additive selected from the group consisting of sodium heparin, lithium heparin, and ethylenediaminetetraacetic acid (EDTA).

7. The method of any of claims 1-5, wherein the at least one detector of the detector arrangement includes a luminometer (520) and wherein the portion of the plasma is analyzed with the luminometer.

8. The method of claim 7, further comprising:
pipetting the portion of the plasma from the single sample tube to a vessel (30) of the integrated automated analyzer with the transporter arrangement;
pipetting a C-Peptide reagent (20) into the vessel with the transporter arrangement;
determining a C-Peptide concentration corresponding to the whole blood sample by applying a reagent capture luminescence method about the vessel with the luminometer; and
reporting a quantitative assessment of an ability of pancreatic beta cells to secrete insulin corresponding to the whole blood sample.

9. The method of any one of claim 7 or 8, wherein the single sample tube comprises a lithium heparin or EDTA additive.

10. The method of claim 9, wherein the additive is lithium heparin and the at least one detector of the detector arrangement includes an ion selective electrode (ISE) (440) for performing electrolyte analysis.

11. The method of claim 7 or claim 8, further comprising:
pipetting the portion of the plasma from the single sample tube to a vessel (30) of the integrated automated analyzer with the transporter arrangement;
pipetting an Insulin reagent (20) into the vessel with the transporter arrangement;
determining an insulin concentration corresponding to the whole blood sample by applying a reagent capture luminescence method about the vessel with the luminometer; and
reporting a quantitative determination of insulin levels corresponding to the whole blood sample.

12. The method of claim 11, wherein the single sample tube comprises an EDTA additive.

13. The method of any of claims 1-12, wherein results from analyzing the portion of the mixed whole blood sample with the at least one detector are automatically used in determining details of analyzing the portion of the plasma with the detector arrangement.

14. The method of any of claims 1-13, wherein the pretreatment module further comprises a capper (290) configured to apply the cap on the single sample tube, the method further comprising:
applying the cap on the single sample tube prior to mixing the whole blood sample in the single sample tube with the whole blood mixer.

15. The method of any of claims 1-13, wherein the pretreatment module further comprises a capper (290) configured to apply the cap on the single sample tube, the method further comprising:
applying the cap on the single sample tube prior to centrifuging the whole blood sample in the single sample tube with the centrifuge.

16. The method of any of claims 1-15, wherein the pretreatment module further comprises a decapper (320) configured to remove the cap from the single sample tube, the method further comprising:
removing the cap from the single sample tube prior to delivering the portion of the mixed whole blood sample from the single sample tube to the at least one detector with the transporter arrangement.

17. The method of any of claims 1-15, wherein the pretreatment module further comprises a decapper (320) configured to remove the cap from the single sample tube, the method further comprising:
removing the cap from the single sample tube prior to delivering the portion of the plasma from the single sample tube to the detector arrangement.

18. The method of any of claims 1-17, wherein the pretreatment module further comprises a reader (390) configured to identify the single sample tube, the method further comprising:
reading an identity of the single sample tube after presenting the whole blood sample to the pretreatment module in the single sample tube.

19. The method of any of claims 1-18, wherein the integrated automated analyzer further comprises at least one rack (70) and wherein the whole blood sample is presented to the pretreatment module of the integrated automated analyzer with the single sample tube in the rack.

20. The method of any of claims 1-18, wherein the pretreatment module further comprises at least one storage location (350) configured to store the single sample tube, the method further comprising:
storing the single sample tube at the storage location after presenting the whole blood sample to the pretreatment module in the single sample tube.

21. The method of claim 20, wherein the single sample tube is stored capped.

22. The method of any of claims 1-18, wherein the pretreatment module further comprises at least one storage location (350) configured to store the single sample tube, the method further comprising:
storing the single sample tube at the storage location after delivering the portion of the mixed whole blood sample from the single sample tube to the at least one detector with the transporter arrangement.

23. The method of any of claims 1-18, wherein the pretreatment module further comprises at least one storage location (350) configured to store the single sample tube, the method further comprising:
storing the single sample tube at the storage location after delivering the portion of the plasma from the single sample tube to the detector arrangement.

24. The method of any of claims 20-23, wherein the integrated automated analyzer further comprises at least one rack (70), the method further comprising:
storing the single sample tube in the rack at the storage location.

25. The method of claim 1 or 2, wherein the at least one detector of the detector arrangement includes an ion selective electrode (ISE) (440) for performing electrolyte analysis.
